(19) United States
(12) Patent Application Publication
(10) Pub. No.: US 2025/0228740 A1
(43) Pub. Date: Jul. 17, 2025

(54) NASAL IRRIGATION DEVICE

(71) Applicant: MICHAEL TODD BEAUTY LP, Port St Lucie, FL (US)

(72) Inventors: Michael Friend, Port St. Lucie, FL (US); Xu Neng Fei, Ninghai Ningbo.ZheJiang (CN); Philip E. Levy, Pittsburgh, PA (US)

(21) Appl. No.: 19/016,845

(22) Filed: Jan. 10, 2025

Related U.S. Application Data
(60) Provisional application No. 63/620,472, filed on Jan. 12, 2024.
(60) Provisional application No. 63/648,842, filed on May 17, 2024.
(60) Provisional application No. 63/666,123, filed on Jun. 29, 2024.
(60) Provisional application No. 63/705,354, filed on Oct. 9, 2024.

(51) Int. Cl. A61H 35/04; A61M 1/00; A61M 3/02; A61M 39/12

(52) U.S. Cl. CPC A61H 35/04; A61M 1/77; A61M 3/0258; A61M 39/12; A61M 2209/10

(57) ABSTRACT

A nasal irrigation device includes a clean liquid tank, a waste liquid tank, a tubing assembly, first and second nasal inserts, a first motorized pump coupled to the clean liquid tank and the tubing assembly structured to draw liquid from the clean liquid tank and pump the liquid to the tubing assembly and to the first nasal insert with positive pressure when the first nasal insert is fixedly or removably coupled to the tubing assembly, and a second motorized pump separate from the first motorized pump and coupled to the waste liquid tank and the tubing assembly structured to draw waste liquid from the tubing assembly and the second nasal insert with negative pressure when the second nasal insert is fixedly or removably coupled to the tubing assembly and provide the waste liquid to the waste liquid tank.

### CROSS REFERENCE TO RELATED APPLICATIONS

This application claims priority to U.S. Provisional Patent Application Ser. No. 63/620,472, filed on Jan. 12, 2024, and titled “Nasal Lavage Devices and Methods of Making and Using Same,” U.S. Provisional Patent Application Ser. No. 63/638,842, filed on May 17, 2024, and titled “Nasal Lavage Devices and Methods of Making and Using Same,” U.S. Provisional Patent Application Ser. No. 63/666,123, filed on Jun. 29, 2024, and titled “Nasal Insert for a Nasal Irrigation Device,” and U.S. Provisional Patent Application Ser. No. 63/705,354, filed on Oct. 9, 2024, and titled “Nasal Insert for a Nasal Irrigation Device,” the disclosures of which is incorporated herein by reference.

### FIELD

The presently disclosed technology relates generally to devices for lavaging, irrigating, rinsing, and/or hydrating the nasal cavity and anatomical openings thereto, generally and popularly known as nasal lavage or nasal irrigation.

More particularly, the presently disclosed technology relates to a compact, portable, rechargeable battery-powered tabletop nasal irrigation device that safely and comfortably washes the nasal cavity with pressurized lightly salted water (“saline solution”) or another liquid, and simultaneously provides powered-suction to remove the liquid waste generated to a waste liquid receptacle incorporated in the device for easy disposal.

### BACKGROUND

Nasal and sinus disease and related conditions and symptoms are a common clinical problem. Bathing the nasal and sinus cavities with saline solution or another liquid to wash away encrusted mucous, irritants, and foreign particles, improve airflow and relieve nasal congestion is well known. Clinical studies demonstrate that regular use of nasal lavage is an effective therapy to relieve the symptoms of chronic and acute rhinosinusitis, including reducing nasal congestion and increasing nasal passage airflow.

Nasal lavage is accomplished by positive pressure (relative to atmospheric pressure) or negative pressure. Positive pressure involves introducing saline solution into the nasal cavity by gravity or manufactured pressure and allowing the effluent generated to drain out the other nostril. Negative pressure, also called vacuum or suction, is achieved anatomically by nasal inhaling or sniffing, or manufactured suction.

Positive pressure devices that rely on gravity, such as the neti pot, allow the flow of saline solution into the nasal cavity by angling the head to the side and pouring the saline solution into one nostril. The saline solution then flows around the posterior margin of the nasal septum and out the user's other nostril. However, gravity-based devices require holding the head in an awkward physical position which can be uncomfortable and difficult for many users. Further, the free-flowing effluent from the nasal passageway is messy so that substantial clean up procedures are necessary.

Positive pressure devices that rely on mechanical pressure via a pump or squeeze bottle inject saline solution into one nostril by squeezing or pumping through the nasal cavity and allowing it to drain out the other nostril. While these devices avoid holding the head in an awkward position, the effluent from the nasal passageway still requires substantial cleanup.

Early nasal lavage devices that relied on negative manufactured pressure were large, non-portable and often complex and difficult to operate. Subsequently, smaller more portable devices were introduced that use negative manufactured pressure to draw a saline solution through the nasal cavity by imparting powered suction directly to the nasal cavity. However, a drawback of these devices is they require the user to hold the device against the face with both hands while in use. Examples of these smaller, more portable devices are shown and described in U.S. Pat. Nos. 7,981,077 and 11,413,377.

Nasal irrigation devices generally comprise a pair of nasal inserts which are held up against the user' nose so that the liquid from the nasal irrigation device can be pumped through the user's nasal cavity. The nasal inserts are connected to ports which are in a fixed position. If the nasal inserts are likewise in a fixed position, users may not be able to effectively engage their nostrils with the nasal inserts because the distance between the nasal openings may be narrower or wider than the fixed position of the nasal inserts. This can prevent the nasal inserts from effectively pushing saline solution or another liquid through the nasal cavity and can result in the liquid from the nasal irrigation device leaking out of the user's nose.

### SUMMARY

In one embodiment, the disclosed concept includes a nasal irrigation device that includes a clean liquid tank, a waste liquid tank, a tubing assembly, a first nasal insert structured to be inserted into a first nostril of a user, the first nasal insert either being fixedly coupled to the tubing assembly or structured to be removably coupled to the tubing assembly, a second nasal insert structured to be inserted into the second nostril of the user, the second nasal insert either being fixedly coupled to the tubing assembly or structured to be removably coupled to the tubing assembly, a first motorized pump coupled to the clean liquid tank and the tubing assembly, the first motorized pump being structured to draw liquid from the clean liquid tank and pump the liquid to the tubing assembly and to the first nasal insert with positive pressure when the first nasal insert is fixedly or removably coupled to the tubing assembly, and a second motorized pump separate from the first motorized pump and coupled to the waste liquid tank and the tubing assembly, the second motorized pump being structured to draw waste liquid from the second nasal insert and tubing assembly and with negative pressure when the second nasal insert is fixedly or removably coupled to the tubing assembly and provide the waste liquid to the waste liquid tank.

In another embodiment, the disclosed concept provides a method of providing nasal irrigation to an individual that includes drawing clean liquid from a clean liquid tank and pumping the clean liquid to a first nostril of the individual with positive pressure generated by a first motorized pump while simultaneously drawing a waste liquid from a second nostril of the individual with negative pressure generated by a second motorized pump separate from the first motorized pump and providing the waste liquid to a waste liquid tank.

### BRIEF DESCRIPTION OF THE DRAWINGS

The following detailed description of the presently disclosed technology will be better understood when read in conjunction with the appended drawings, wherein like numerals designate like elements throughout. For the purpose of illustrating the presently disclosed technology, there are shown in the drawing's various illustrative embodiments. It should be understood, however, that the presently disclosed technology is not limited to the precise arrangements and instrumentalities shown. In the drawings:

FIG. 1 is a perspective view of the nasal irrigation device according to an optional aspect of the presently disclosed technology;

FIG. 2 is a front elevational view of the nasal irrigation device shown in FIG. 1;

FIG. 3 is a side elevational view of the nasal irrigation device shown in FIG. 1;

FIG. 4 is a rear elevational view of the nasal irrigation device shown in FIG. 1;

FIG. 5 is a top plan view of the nasal irrigation device shown in FIG. 1;

FIG. 6 is an exploded view of the nasal irrigation device shown in FIG. 1;

FIGS. 7 and 8 are cross-sectional views of the nasal irrigation device shown in FIG. 1, taken along lines A-A of FIG. 5 showing certain selected internal components of the nasal irrigation device (in an embodiment without the optional storage housing);

FIG. 9 is a cross-sectional view of the nasal irrigation device shown in FIG. 1, taken along lines B-B of FIG. 5;

FIG. 10 is a front elevational view of the nasal irrigation device of FIG. 1 with a cover installed on or over at least a portion of the tubing assembly;

FIG. 11 is a front view of a pair of nasal inserts of a nasal irrigation device according to an exemplary embodiment of the disclosed concept;

FIG. 12 is a front view of a handheld tubing assembly of a nasal irrigation device according to an exemplary embodiment of the disclosed concept that includes the nasal inserts of FIG. 11;

FIG. 13 is a cross-sectional view of the nasal insert of FIG. 11 taken along axis X-X of FIG. 11;

FIG. 14 is a front view of a pair of nasal inserts and a portion of a handle according to an exemplary embodiment;

FIG. 15 is a perspective view of the nasal insert and handle shown in FIG. 14; and

FIG. 16 is an isometric view of a self-cleaning tool for the nasal irrigation device of the disclosed concept according to an exemplary embodiment.

### DETAILED DESCRIPTION

While systems, devices and methods are described herein by way of examples and embodiments, those skilled in the art will recognize that the presently disclosed technology is not limited to the embodiments or drawings described. Rather, the presently disclosed technology covers all modifications, equivalents and alternatives falling within the spirit and scope of the appended claims. Features of any one embodiment disclosed herein can be omitted or incorporated into another embodiment.

Any headings used herein are for organizational purposes only and are not meant to limit the scope of the description or the claims. As used herein, the word “may” is used in a permissive sense (i.e., meaning, having the potential to) rather than the mandatory sense (i.e., meaning, must). Unless specifically set forth herein, the terms “a,” “an” and “the” are not limited to one element but instead should be read as meaning “at least one.” The terminology includes the words noted above, derivatives thereof and words of similar import.

As used herein, “and/or” means that either or both of the items separated by such terminology are involved. For example, the phrase “A and/or B” should be read to mean A alone, B alone, or both A and B.

As used herein, “generally” means “in a general manner” relevant to the term being modified as would be understood by one of ordinary skill in the art.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, “fixedly coupled” or “fixed” means that two components are coupled so as to move as one while maintaining a constant orientation relative to each other.

As used herein, the statement that two or more parts or components “engage” one another shall mean that the parts exert a force against one another either directly or through one or more intermediate parts or components.

As used herein, the term “number” shall mean one or an integer greater than one (i.e., a plurality).

Directional phrases used herein, for example and without limitation, top, bottom, left, right, upper, lower, front, back, and derivatives thereof, relate to the orientation of the elements shown in the drawings and are not limiting upon the claims unless expressly recited therein.

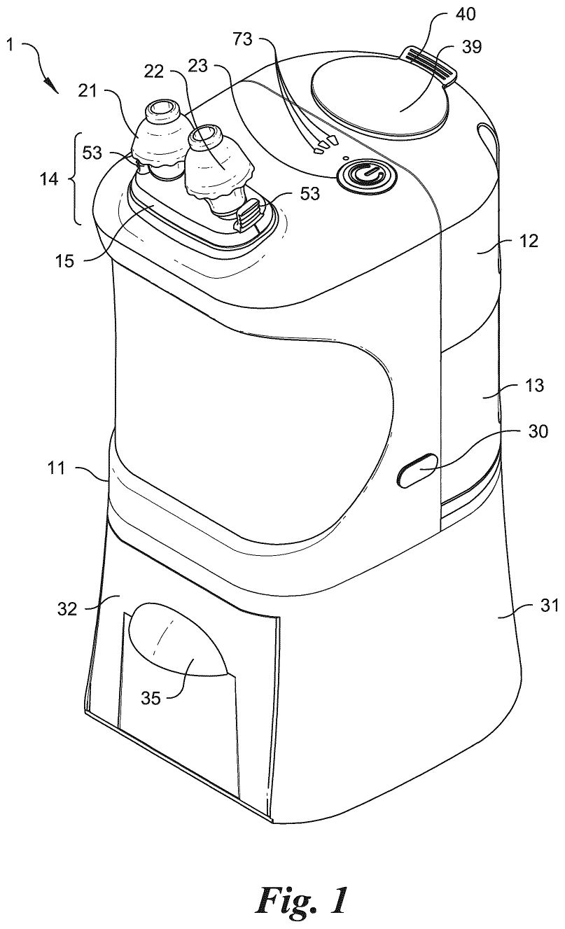

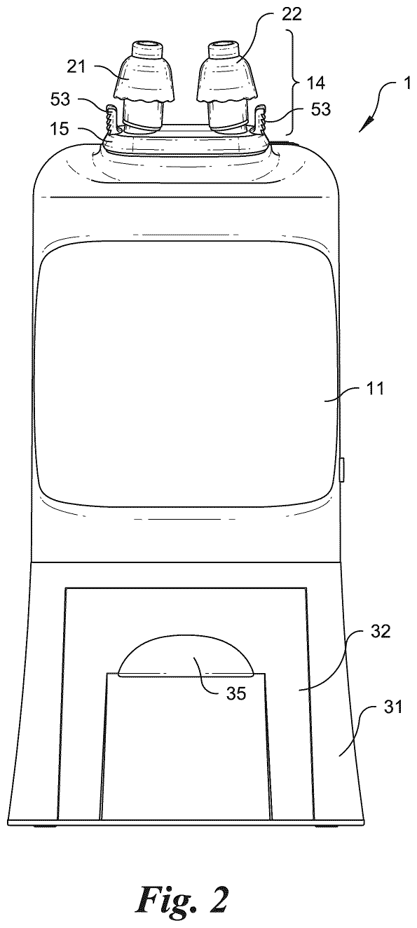

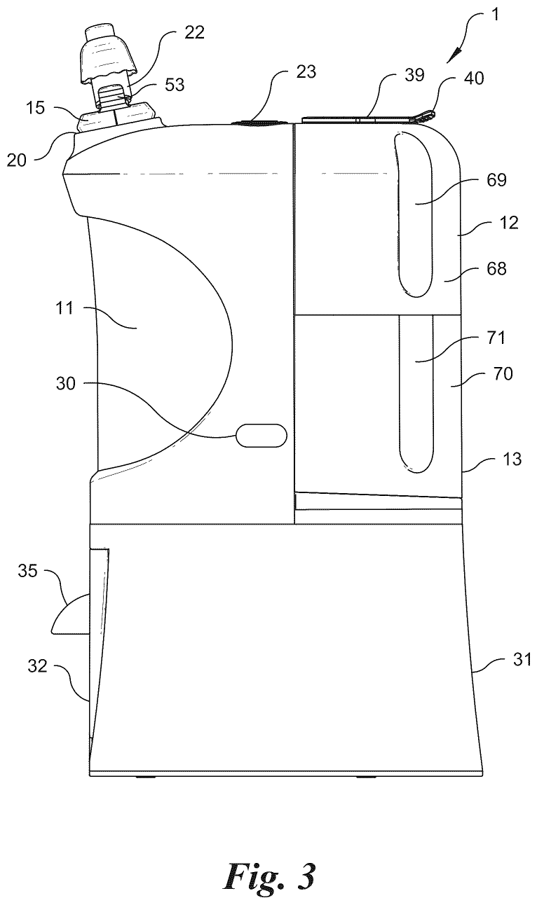

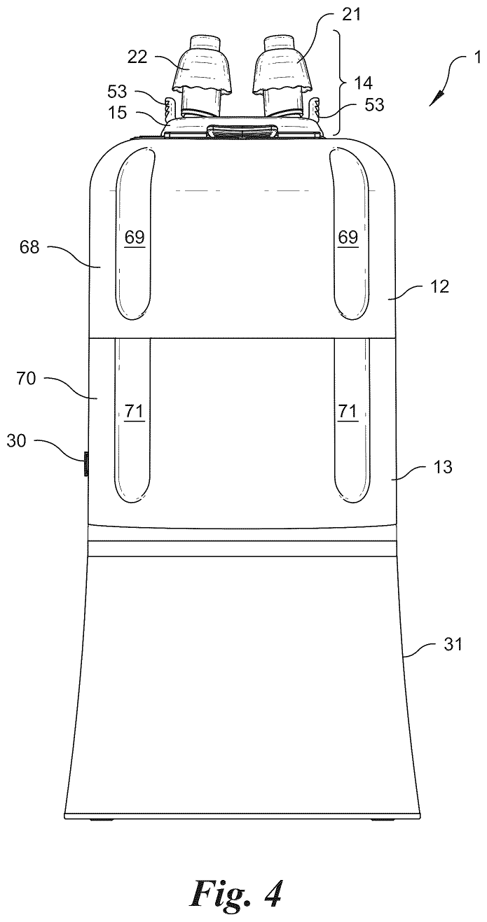

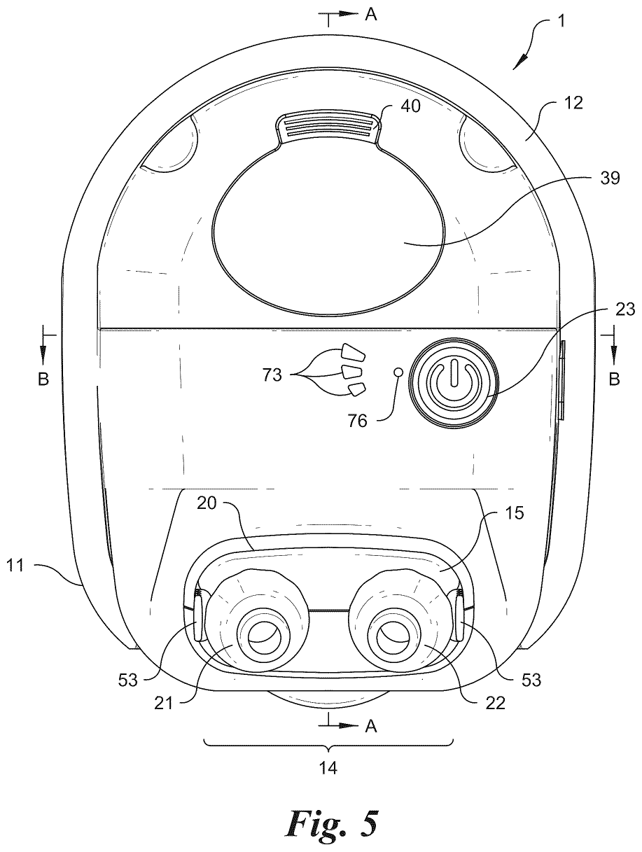

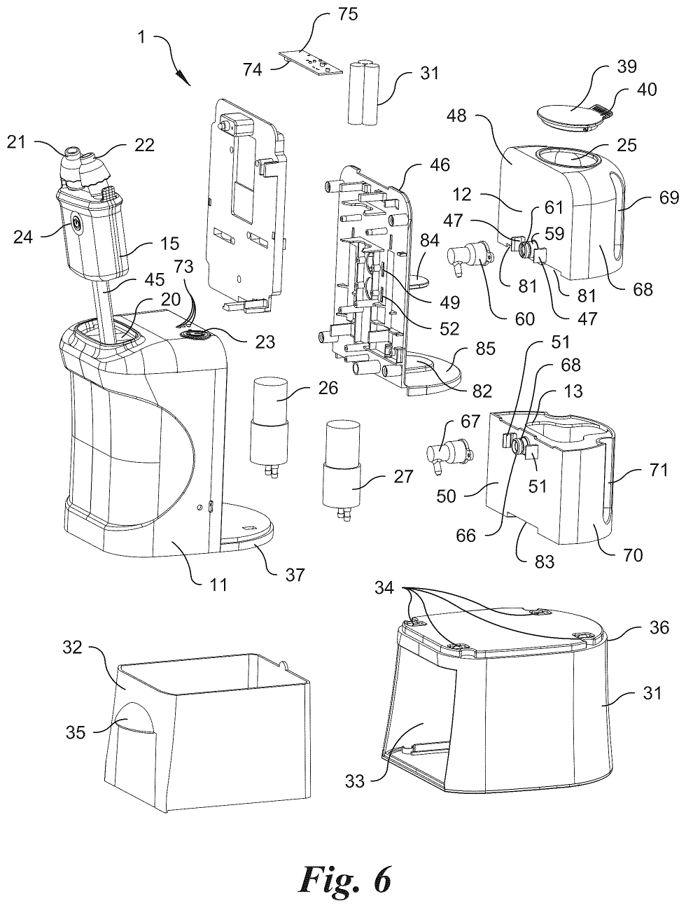

FIG. 1 is a perspective view, FIG. 2 is a front elevational view, FIG. 3 is a side elevational view, FIG. 4 is a rear elevational view, and FIG. 5 is a top plan view of a nasal irrigation device 1 according to an exemplary embodiment of the presently disclosed technology. FIG. 6 is an exploded view of the nasal irrigation device 1. The nasal irrigation device 1, as shown, includes a main housing 11. The main housing 11 includes a lower surface disposed opposite an upper surface with a sidewall defined in between. The lower surface can be flat or planar, and is configured to support the weight of the main housing 11 on a flat surface, such as a table or countertop. This design is intended to promote an almost hands-free use of the nasal irrigation device 1, such that the user is not required to hold or lift-up the entire device for use.

The main housing 11 defines an interior volume to house one or more components of the nasal irrigation device 1 described elsewhere herein. The main housing 11 is configured to removably receive a clean liquid tank 12 and a waste liquid tank 13. In the demonstrated embodiment, the clean liquid tank 12 is in a vertically elevated position relative to the waste liquid tank 13. However, other arrangements of the tanks 12, 13, such as laterally side-by-side, are also possible.

As seen in the exploded view shown in FIG. 6, the rear wall 46 of main housing 11 includes a one or more platforms or shelves 84 on which the clean liquid tank 12 may rest when connected to the main housing 11 and a platform or surface 85 on which the waste liquid tank 13 may rest when connected to the main housing 11. In the exemplary embodiment, two spaced apart shelves 84 are provided for this purpose and a single flat surface 85 is provided for this purpose. In addition, the rear wall 46 of main housing 11 includes a pair of slots 49 for receiving a pair of tabs 47 that are provided on a front wall 48 of the clean liquid tank 12. Similarly, the rear wall 46 of the main housing 11 includes a pair of slots 52 for receiving a pair of tabs 51 that are provided on a front wall 50 of waste liquid tank 13. Also, the bottom of the clean liquid tank 12 includes a number of indentions 81 that each have a shape that is completely to the shape of shelves 84 for receiving the shelves 84 to facilitate the coupling of the clean liquid tank 12 to the housing 11. Similarly, the surface 85 includes a raised portion 82 and the waste liquid tank 13 includes an indention 83 that has a shape that is completely to the shape of the raised portion 82 for receiving raised portion 82 to facilitate the coupling of the waste liquid tank 13 to the housing 11. In addition, in an exemplary embodiment, the bottom internal surface of the clean water tank 12 is tilted relative to the plane of the bottom of the main housing 11 and specifically the surface 85. This tilt facilitates the flow of liquid from the clean water tank 12 into the main housing 11 through the sealing assembly 58. In one particular exemplary embodiment, the bottom internal surface of the clean water tank 12 lies in a plane that is tilted 2° relative to the plane of the bottom of the main housing 11 (that is, there is an angle of 2° between the plane in which the bottom internal surface of the clean water tank 12 lies and the plane in which the bottom internal surface of the clean water tank 12 lies).

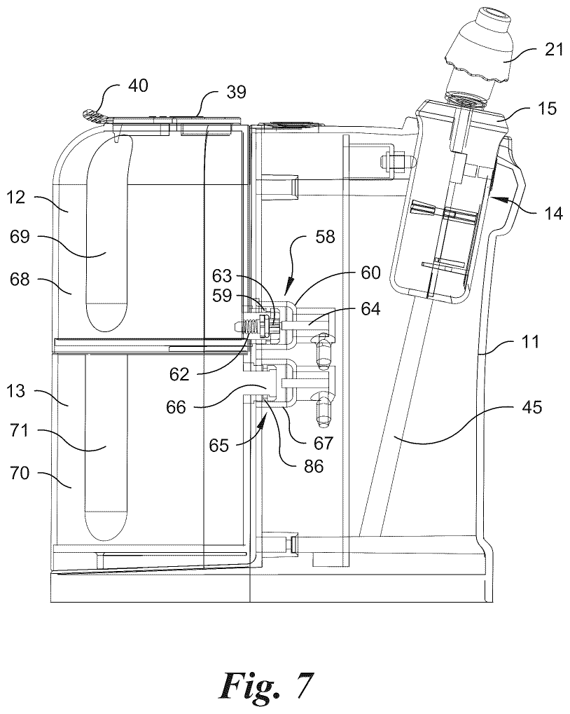

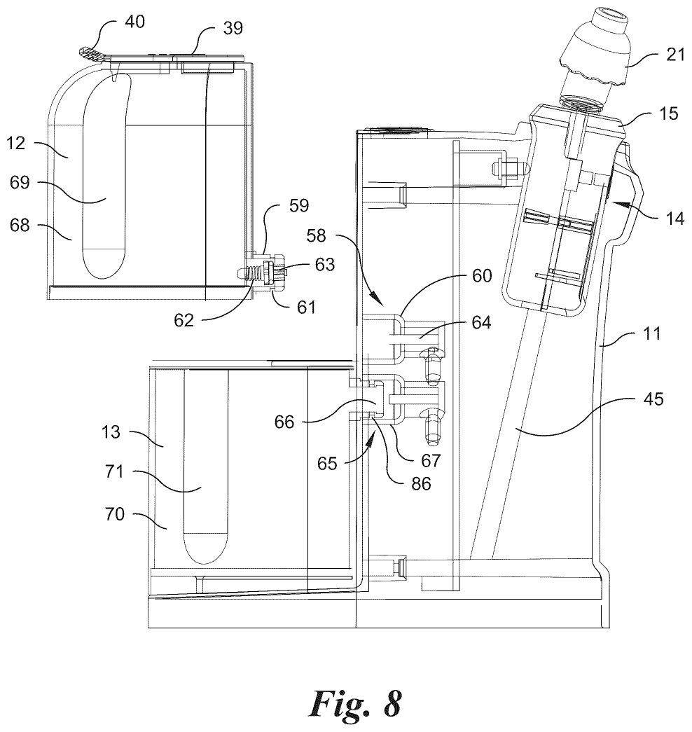

FIGS. 7 and 8 are cross-sectional views of the nasal irrigation device 1 taken along lines A-A of FIG. 5 showing certain selected internal components of the nasal irrigation device (in an embodiment without the optional storage housing 31 that is described herein). In FIG. 8, the clean liquid tank 12 is separated from the main housing 11, such as would be the case when the clean liquid tank 12 is being filled with saline solution or another liquid.

As seen in FIGS. 6-8, the clean liquid tank 12 includes a sealing assembly 58 that comprises a mechanism for coupling the clean liquid tank 12 to the main housing 11. The sealing assembly 58 includes a cylindrically shaped male fluid coupling member 59 that extends from the front wall 48 of the clean liquid tank 12. The male fluid coupling member 59 is structured to be received within a female fluid coupling member 60 that is provided at least partially within the main housing 11. The female fluid coupling member 60 is coupled to a motorized positive pressure rinsing pump 26 (described herein) that is provided within the main housing 11. A sealing ring 61 is provided on the outer surface of the male fluid coupling member 59 to create a fluid tight seal when the male fluid coupling member 59 is received within the female fluid coupling member 60. In addition, a spring 62 and an activity pole 63 are provided within the male fluid coupling member 59. The spring 62 biases the activity pole 63 against a sealing surface provided at the terminal end of the male fluid coupling member 59 such that the male fluid coupling member 59 is closed and does not allow saline solution or another liquid to pass outwardly from the clean liquid tank 12 when the male fluid coupling member 59 is in this biased condition. A post member 64 is provided within the female fluid coupling member 60. When the male fluid coupling member 59 is received within the female fluid coupling member 60, the post member 64 engages the activity pole 63 and pushes it against the spring 62, causing the spring 62 to compress and the activity pole 63 to move away from the sealing surface. As a result, saline solution or another liquid is able to pass from the clean liquid tank 12 through the male fluid coupling member 59 and the female fluid coupling member 60 and into the positive pressure rinsing pump 26 so that it can be pumped to the nasal passages of the user as described herein.

As also seen in FIGS. 6-8, the waste liquid tank 13 includes a sealing assembly 65 that comprises a mechanism for coupling the waste liquid tank 13 to the main housing 11. The sealing assembly 65 includes a cylindrically shaped male fluid coupling member 66 that extends from the front wall 50 of the waste liquid tank 13. The male fluid coupling member 66 is structured to be received within a female fluid coupling member 67 that is provided within the main housing 11. The female fluid coupling member 67 is coupled to a motorized negative pressure suction pump 27 (described herein) that is provided within the main housing 11. A sealing ring 86 is provided on the outer surface of the male fluid coupling member 66 to create a fluid tight seal when the male fluid coupling member 66 is received within the female fluid coupling member 67. As a result, waste liquid is able to pass from the negative pressure suction pump 27 through the female fluid coupling member 67 and the male fluid coupling member 66 and into waste liquid tank 13 as described herein. The positive pressure rinsing pump and the negative pressure suction pump may be any type of suitable motorized pump, such as, without limitation, a positive displacement pump (for example a diaphragm pump) or a centrifugal pump.

In addition, the clean liquid tank 12 includes an aperture 25 for filling the clean liquid tank 12 with clean saline solution or another liquid. A lid 39 is provided for covering the aperture 25. The lid is structured to be friction/snap fit onto the clean liquid tank 12 within the aperture 25 to cover the aperture 25 after it is filled with saline solution or another liquid. The lid 39 includes an upwardly extending and angled tab member 40. The lid 39 is able to be removed from the aperture 25 by lifting the tab member 40 or exerting a downward force on the tab member 40, which force in either case overcomes the snap/friction fit with aperture 25 (i.e., the edges of the clean liquid tank 12 forming the aperture 25). Furthermore, the clean liquid tank 12 has a semicircular-shaped rear wall 68 having a pair of elongated semicircular-shaped indentations 69 for receiving the user's thumb and fingers therein to facilitate the coupling and de-coupling of the clean liquid tank 12 to and from the main housing 11. Similarly, the waste liquid tank 13 has a semicircular-shaped rear wall 70 having a pair of elongated semicircular-shaped indentations 71 for receiving the user's thumb and fingers therein to facilitate the coupling and de-coupling of the waste liquid tank 13 to and from the main housing 11.

The main housing 11 can rest upon an optional storage housing 31 (the nasal irrigation device 1 can be used with or without the optional storage housing 31). The storage housing 31 may be entirely separable from the main housing 11, and the main housing 11 may be used without the storage housing 31. An outer periphery of the main housing 11 transitions smoothly and gradually into the outer periphery of the storage housing 31, thereby giving the appearance of a single, unitary unit.

The storage housing 31 includes at least one drawer 32, which is insertable, movable within, and/or removable from a storage housing opening 33. As shown in FIG. 6, the drawer 32, which can have a generally rectilinear shape, can occupy a majority of the interior area, or a significant portion thereof, of the storage housing 31. For example, an exterior height and width of the drawer 32 can be only slightly less than an exterior height and width of the storage housing 31. The drawer 32 defines a storage compartment. The drawer 32 includes four sidewalls and a bottom wall, thereby giving the drawer 32 an open top.

The storage housing 31 provides additional storage for the nasal irrigation device 1 and/or can increase the height at which a top of the main housing 11 rests relative to a user and/or a support surface on which the nasal irrigation device 1 rests. One or more product accessories, such as nasal inserts and salt packets, may be stored within the drawer 32 so that they are conveniently accessible to the user of the nasal irrigation device 1 and/or are protected from damage or being misplaced. The drawer 32 may optionally be configured to define a liquid tight, or a substantially liquid tight, seal around the storage housing opening 33, to prevent liquid damage to the product accessories stored inside the drawer 32.

In the illustrated embodiment, the drawer 32 comprises at least one drawer handle 35 disposed on a front face of the drawer 32 and extending laterally outwardly therefrom. The drawer handle 35 is configured to assist the user in opening the drawer 32 and can form a cavity thereunder for insertion of one or more fingers of a user. In the exemplary embodiment, the drawer handle 35 is arcuate in two separate planes, and is located in the middle of the front face of the drawer 32.

The storage housing 31 includes a flat bottom surface, such that the nasal irrigation device can rest securely on a flat surface, such as a counter or tabletop, when the main housing 11 is placed upon the storage housing 31. As seen in FIG. 6, the storage housing 31 also includes one or more spaced-apart recesses 34 (see FIG. 6 in a top surface thereof). The one or more recesses are dimensioned to receive one or more legs or projections of the main housing 11, so that when the one or more legs are inserted into the corresponding recesses 34, movement or shifting of the main housing 11 relative to the storage housing 31 will be eliminated or at least minimized. In the illustrated embodiment, the storage housing 31 defines a storage housing ledge 36 to receive at least a portion of downward extending sidewall 37 from the base of the main housing 11. As such, the space between the storage housing 31 and the main housing 11 will be closed when the main housing 11 is placed on the storage housing 31. This creates a stable, secure connection between the storage housing 31 and the main housing 11.

Furthermore, the nasal irrigation device 1 includes a tubing assembly 14 that is coupled to and partially stored within a compartment 72 provided in the front of the main housing 11 that is accessible by way of an opening 20 that is provided in the top of the main housing 11. The tubing assembly 14 is configured to provide an improved almost hands-free (i.e., fingertips of a single hand) operation of the nasal irrigation device 1. In contrast to prior art systems, which require the user to lift the entire device to the nose and hold it up against the nostrils in order to operate the system, the nasal irrigation device 1 allows the main housing 11 to remain on a support surface (e.g., a countertop or table top) while the tubing assembly 14 is extended from the main housing 11 to reach the user's nose. Specifically, as described in more detail below, at least a portion of the tubing assembly 14 can be lifted upwardly out of the compartment 72 and away from the main housing 11 toward or to the user's nose. Thus, the system of the presently disclosed technology is adjustable, extendable, and retractable to accommodate the user and to facilitate ease of use.

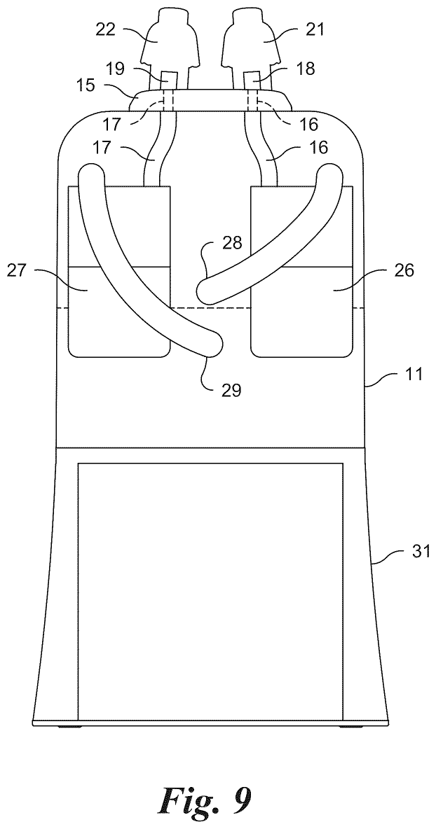

FIG. 9 is a cross-sectional view of the nasal irrigation device shown 1 taken along line B-B of FIG. 5, which shows certain selected internal components of the nasal irrigation device 1. To accomplish the above-described functionality of the tubing assembly 14, the tubing assembly 14 includes a housing comprising a base or handle 15, a first tube 16, and a second tube 17. Preferably, the first tube 16 and the second tube 17 are contained within a single tube sheath 45. The first tube 16 and the second tube 17 are coupled to the bottom end of the handle 15. The handle 15 is removably securable within and movable with respect to the orifice 20 and the compartment 72 of the main housing 11. The first tube 16 and the second tube 17 are of sufficient length (i.e., can include slack) to remain attached to the necessary structure while accommodating movement of the handle 15 as described herein.

In addition, as shown in FIG. 9, the top end of the handle 15 includes a first port member 18 and a second port member 19 extending from the top end of the handle 15. The handle 15 also includes a first nozzle or nasal insert 21 removably disposed on or attached to the first port member 18 and a second nozzle or nasal insert 22 removably disposed on or attached to the second port member 19. The first nasal insert 21 and the second nasal insert 22 can each be configured to be removably inserted at least partially into a first nostril and a second nostril, respectively, of the user.

The upper or proximal end of the first tube 16 is received within the handle 15 and is fluidly coupled to the first port member 18. The lower or distal end of the first tube 16 is fluidly coupled to an output of the positive pressure rinsing pump 26. The input of the positive pressure rinsing pump 26 is fluidly coupled to a tube 28 at a first end thereof. The other end the tube 28 is fluidly coupled to the female fluid coupling member 60 and the male fluid coupling member 59, which in turn are fluidly coupled to the clean liquid tank 12 as described herein. As a result, the positive pressure rinsing pump 26 is configured for pumping saline solution or another liquid from the clean liquid tank 12 to the nozzle 21 with positive pressure via the path just described.

The upper or proximal end of the second tube 17 is also received within the handle 15 and is fluidly coupled to the second port member 19. The lower or distal end of the second tube 17 is fluidly coupled to an input of the negative pressure suction pump 27. The output of the negative pressure suction pump 27 is fluidly coupled to a tube 29 at a first end thereof. The other end the tube 29 is fluidly coupled to the female fluid coupling member 67 and the male fluid coupling member 66, which in turn are fluidly coupled to the waste liquid tank 13 as described herein. As a result, the negative pressure suction pump 27 is configured for suctioning waste liquid from by the nozzle 22 with negative pressure to the waste liquid tank 13 via the path just described.

In addition, as seen in FIG. 6, the handle 15 includes an actuator button 24 on the front face thereof. The actuator button 24 is configured to control operation of the positive pressure rinsing pump 26 and the negative pressure suction pump 27. In particular, when the nasal irrigation device 1 is in the powered-on condition as a result of actuation of button 23, pressing and holding of the actuator button 24 activates the positive pressure rinsing pump 26 and the negative pressure suction pump 27 at the power level set by the user via button 23 and indicated by lights 73. Releasing of the actuator button 24 de-activates the positive pressure rinsing pump 26 and the negative pressure suction pump 27 to pause or end treatment.

In one exemplary embodiment, the first nasal insert 21 and the second nasal insert 22 are made of a silicone material. In yet another embodiment, the first hose 16 and the second hose 17 may be automatically retractable, so as to assist the user with placing the tubing assembly 14 into the orifice 20 and the chamber 72 when the nasal irrigation device 1 is not in use. If the first hose 16 and the second hose 17 are automatically retractable, a user may simply manually retract the first hose 16 and the second hose 17 into the main housing 11. In a further embodiment, the first nasal insert 21 and the second nasal insert 22 are rotatable relative to and removable from the first port member 18 and the second port member 19, respectively.

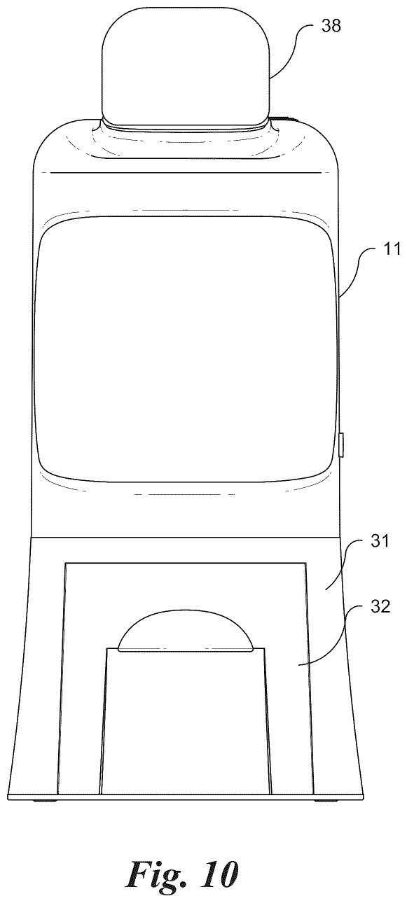

Referring to FIG. 10, the nasal irrigation device 1 can optionally include a nasal insert cover 38. The nasal insert cover 38 is configured to be removably placed over the first nasal insert 21 and the second nasal insert 22 to reduce contamination of the first nasal insert 21 and the second nasal insert 22 and/or protect the first nasal insert 21 and the second nasal insert 22 from damage. In the illustrated embodiment, the handle 15 of the tubing assembly 14 comprises a handle ledge on which a rim of the nasal insert cover 38 can rest when placed over the first nasal insert 21 and the second nasal insert 22. The nasal insert cover 38 can be made of a transparent or translucent material, or an opaque material. The material can be flexible, resilient, and/or pliable.

As described herein, the nasal irrigation device 1 employs two separate electrically powered motorized pumps, specifically the positive pressure rinsing pump 26 and the negative pressure suction pump 27. As such, the nasal irrigation device 1 is bi-powered. In particular, as described above, the first tube 16 is fluidly coupled to the positive pressure rinsing pump 26. When activated, the positive pressure rinsing pump 26 is configured to move saline solution or another liquid from the clean liquid tank 12 through the first tube 16 and the first port member 18 and out of the first nasal insert 21. Such action by the positive pressure rinsing pump 26 generates a powered washing or rinsing effect via positive manufactured pressure of the contents of the clean liquid tank 12 through the first nasal insert 21. In one embodiment, the positive manufactured pressure through the first nasal insert 21 is adjustable, such that more than one defined pressure level may be selected by the user to tailor treatment to specific conditions, comfort levels, and other needs. The second tube 17 is fluidly coupled to the negative pressure suction pump 27. The negative pressure suction pump 27 is configured to move waste liquid via powered suction (i.e., negative manufactured pressure) to move waste liquid from the second nasal insert 22 through the second port member 19 into the proximal end of the second tube 17 and into the waste liquid tank 13. In one embodiment, the negative pressure through the second nasal insert 22 is adjustable, such that more than one defined pressure level may be selected by the user to tailor treatment to specific conditions, comfort levels, and other needs.

In one embodiment, the positive manufactured pressure through the first nasal insert 21 is independently adjustable from the negative manufactured pressure through the second nasal insert 22. In another embodiment, the positive manufactured pressure through the first nasal insert 21 and the negative manufactured pressure through the second nasal insert 22 are synchronized and simultaneously adjustable.

In one embodiment, the positive pressure rinsing pump 26 and the negative pressure suction pump 27 are jointly and simultaneously operated. That is, the positive pressure rinsing pump 26 generates a power washing effect through the first nasal insert 21 while the negative pressure suction pump 27 generates powered suction through the second nasal insert 22. In an alternative embodiment, the negative pressure suction pump 27 may be configured with a predetermined delayed stop protocol relative to the positive pressure rinsing pump 26. As such, when the actuator 24 is pressed and held, the positive pressure rinsing pump 26 is activated and will generate a power washing effect at the power level selected by the user via the button 23 and the negative pressure suction pump 27 will generate powered suction at the power level selected by the user via the button 23. When the actuator 24 is released, the positive pressure rinsing pump 26 will be deactivated and will stop generating the power wash effect, but the negative pressure suction pump 27 will continue to generate powered suction for a predetermined time period, such as a time period between 3 second and 10 seconds. For example, the time period may be 5 seconds. Optionally, the time period may be adjusted by the user.

In yet another alternative embodiment, the positive pressure rinsing pump 26 and the negative pressure suction pump 27 are independently operable. That is, the positive pressure rinsing pump 26 may be started or stopped independently from the negative pressure suction pump 27.

In order to control the operation of the nasal irrigation device 1 as described herein, a control system 74 (including a controller and memory) is provided on a printed circuit board 75 that is provided within the main housing 11. The controller 74 is operatively coupled to the button 23, the actuator 24, the positive pressure rinsing pump 26, and the negative pressure suction pump 27. The control system 74 is provided with a number of software routines for implementing the functionality described herein.

In one optional embodiment, at least one of the clean liquid tank 12, the first tube 16, the second tube 17, the first port member 18, the second port member 19, the first nasal insert 21, the second nasal insert 22, and/or any other component coming into contact with the saline solution or waste liquid is treated with antimicrobial protection, which may be built-in to the material or a coating. Such antimicrobial protection may include any of a number of different active ingredients such as, without limitation, silver ion technology (wherein silver ions are incorporated as an additive at the time of manufacture, or applied as a coating after manufacture), which forms a safe protective antimicrobial surface to resist and inhibit bacterial growth by up to 99.94%. Such active ingredients may further include, without limitation, essential plant oils (e.g., cinnamon oil, clove oil, eucalyptus oil, oregano oil, lavender oil, leleshwa oil, lemon oil, lemon myrtle oil, mint oil, neem oil, nigella sativa (black cumin) oil, peppermint oil, sandalwood oil, ironwort oil, tea tree oil, and thyme oil), and essential minerals, or bacteria resistant polymers (e.g., a polymer that reduces the ability of pathogenic bacteria to attach to the polymer).

The nasal irrigation device 11 further includes a power source 31, such as to enable electronic operation of the nasal irrigation device 1 as described herein, including the powering of the control system 74, the lights 73, the positive pressure rinsing pump 26, and the negative pressure suction pump 27. In one embodiment, the power source 31 is an internal, rechargeable battery connected to a charging port 30 to receive charging power from an external source. For example, the charging port 30 in one embodiment is a universal serial bus (USB) charging port 30 (e.g., USB-A or USB-C).

In operation (according to one exemplary embodiment), a user first fills the clean liquid tank 12 with saline solution or another liquid. Such a saline solution may be created by adding un-iodized salt to distilled, boiled or other purified water in the clean liquid tank 12. The user then powers the nasal irrigation device 1 on by pressing the power button 23. A green pilot light 76 will come on, indicating that the nasal irrigation device 1 is ready to use. In addition, a first one of the lights 73 will come on, indicating that the nasal irrigation device 1 is in the lowest power setting (the gentle cleansing mode). The nasal irrigation device 1 can be switched to either of the two higher power settings (indicated by second and third ones of the lights 73) with subsequent presses of button 23. The user then removes the handles 15 and the nasal tubing assembly 14 from the main housing 11 and extends the handle 15 and the nasal tubing assembly 14 with either hand to the user's nose and inserts the first nasal insert 21 and the second nasal insert 22 into the user's nostrils.

The user then initiates cleansing by pressing and holding actuator 24 on the handle 15. In response, the positive pressure rinsing pump 26 and the negative pressure suction pump 27 will be activated (each at the selected power setting). As a result, saline solution or another liquid will be pumped via the positive manufactured pressure of the positive pressure rinsing pump 26 and out of the clean liquid tank 12 and into and through the first nasal insert 21 into a first nostril of the user. The saline solution or other liquid is simultaneously sucked out of the user's nasal cavity via the negative manufactured pressure of the negative pressure suction pump 27 through the second nostril of the user and into the second nasal insert 22 (the saline being “waste liquid” at this point). The waste liquid that is received in the second nasal insert 22 flows through the second tube 17 and into the waste liquid tank 13, where it can later be disposed of. The user may pause or stop the cleansing at any time by releasing the actuator 24, which will deactivate the positive pressure rinsing pump 26 and the negative pressure suction pump 27, thereby stopping the flow of saline solution or another liquid. The user may reverse the direction of flow by simply turning the handle over and inserting the first nasal insert 21 and the second nasal insert 22 into the opposite nostrils. Once the cleansing procedure is complete, the user may empty the waste liquid from the waste liquid tank 13.

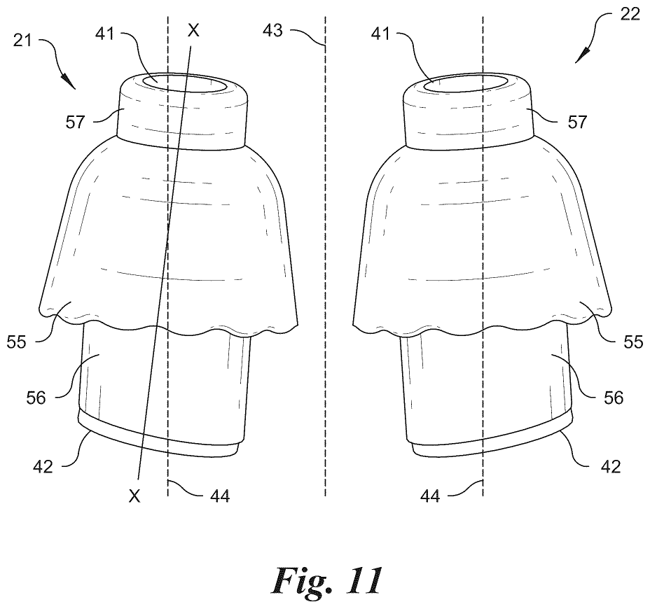

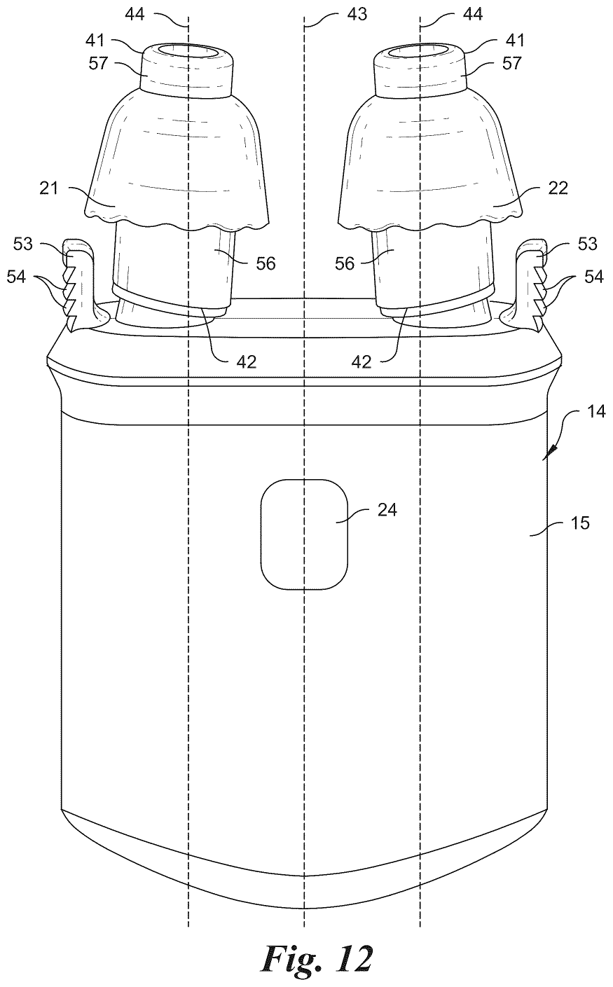

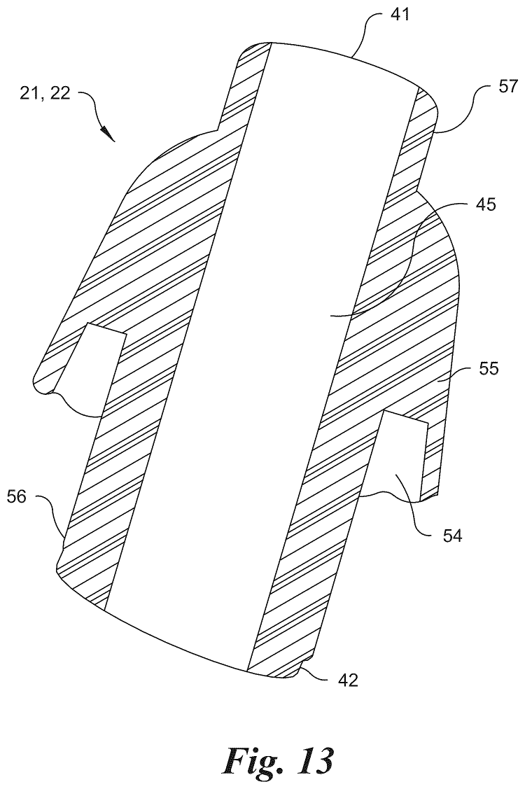

Referring to FIGS. 11 and 12, the first nasal insert 21 and the second nasal insert 22 according to the exemplary embodiment each comprise an aperture 41. Each aperture 41 is disposed at the distal end of the respective nasal insert 21, 22, opposite a base 42, and defines a channel 45 (FIG. 13) through the nasal insert 21, 22. In the illustrated embodiment, the base 42 is an angled base 42. In the exemplary embodiment, the angled base 42 may be cut at a 45 degree angle. The angled base 42 is configured to define the direction in which aperture 41 is directed. Furthermore, the angled based 42 allows the distance between each aperture 41 to be increased or decreased relative to a median line 43 between the first nasal insert 21 and the second nasal insert 22 by rotating the nasal insert 21, 22 in a clockwise or counterclockwise direction around a vertical axis 44 extending centrally through the nasal insert 21, 22 (the base 42 is angled with respect to a normal to that vertical axis 44). As such, the distance between the first nasal insert 21 and the second nasal insert 22 (and specifically the apertures 41 thereof) can be selectively increased or decreased to accommodate the specific size of the user's nose.

In the illustrated embodiment, the first nasal insert 21 and the second nasal insert 22 each include an elongated body having a proximal portion 56 and a distal portion 57 located above the proximal portion 56. The proximal portion 56 includes a cylindrical bottom and a skirt 55 that is located adjacent to and that extends downwardly from the distal portion 57. In the exemplary embodiment, the distal portion 57 has a cylindrical shape, wherein the diameter of the distal portion 57 is smaller than the diameter of the cylindrical bottom of the proximal portion 56. The skirt 55 extends radially around the elongated body and extends outwardly therefrom in an increasing diameter from a top portion of the skirt 55 to a bottom portion of the skirt 55. As such, the skirt 55 is configured to form a seal around the nostril of the user, such that liquid may be efficiently pumped into the nostril (from the first nasal insert 21) or drained from the nostril (from the second nasal insert 22). In the exemplary embodiment, as shown in FIG. 13, the skirt 55 defines an annular space 54 at least partially between the skirt 55 and the cylindrical bottom of the proximal portion 56. The annular space 54 creates flexibility in the skirt 55, such that the user may press the skirt 55 partially inward to assist in rotating the nasal insert 21, 22. With the increased elasticity and flexibility of the skirt 55 provided by the annular space 54, the nasal insert 21, 22 is more easily able to form to the user's nostril. Furthermore, the annular space 54 results in a nasal insert 21, 22 that can be made cheaper as less material is used. The annular space 54 also allows for harder materials to be used to form the nasal inserts 21, 22 which may increase the longevity of the nasal inserts 21, 22.

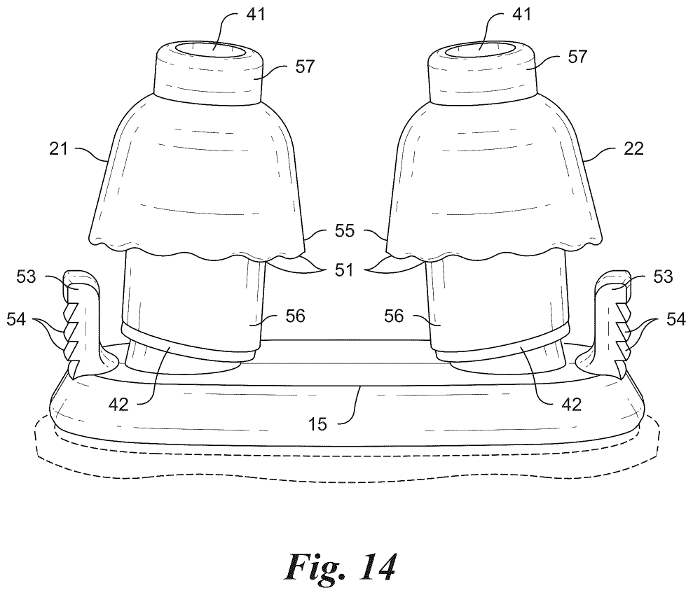

Referring to FIG. 14, in each nasal insert 21, 22, the bottom edge comprises a plurality of downwardly extending scallops 51. In the exemplary embodiment, the plurality of scallops 51 are each curved (e.g., semicircular) and are radially disposed around the bottom edge of the skirt 55. In the exemplary embodiment, each scallop 51 is the same size and shape.

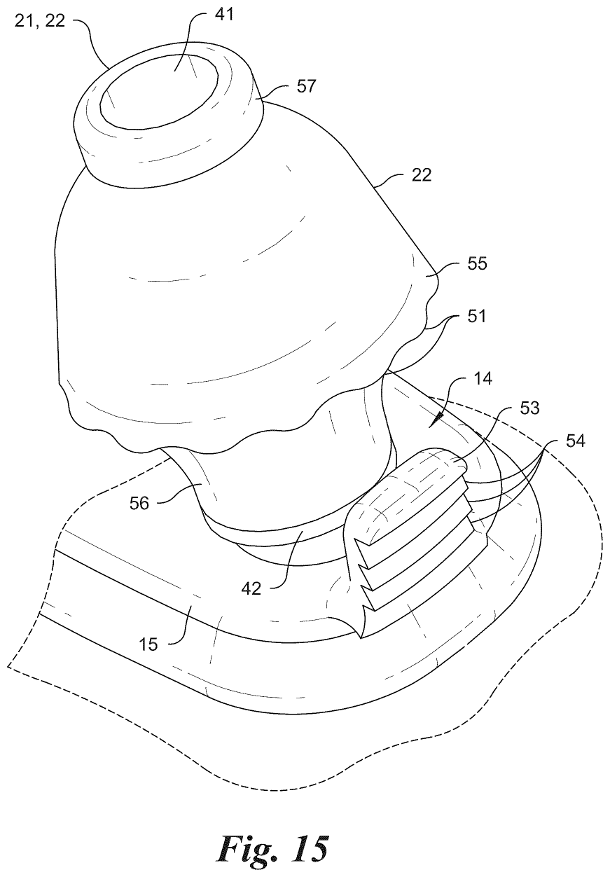

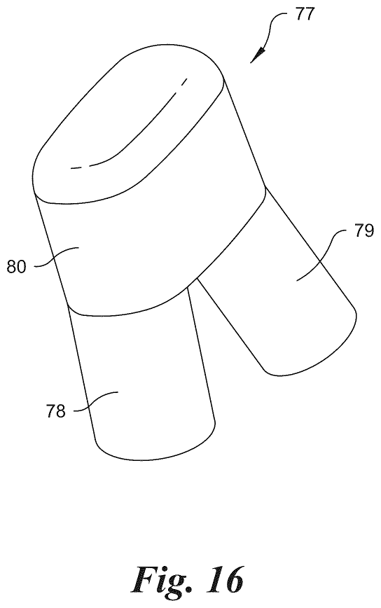

As seen in FIGS. 12, 14 and 15, the handle 15 comprises a pair of grips 53. The grips 53, as shown, extend upwardly from opposing sides of the top surface of the handle 15. The grips 53 are configured to assist users in holding the handle 15 and the tubing assembly 14 while removing it from the orifice 20 and the compartment 72. In the exemplary embodiment, each of the grips 53 comprises a plurality of teeth 54 extending outwardly from an external side surface of each grip 53. In the exemplary embodiment, each of the teeth 54 is elongated. Furthermore, in the exemplary embodiment, each of the teeth 54 is horizontally oriented and is in parallel alignment with the other teeth 54. The plurality of teeth 54 are configured to provide an improving gripping surface for the pair of grips 53.

In the exemplary embodiment, the nasal irrigation device 1 has a self-cleaning capability. In particular, the nasal irrigation device 1 includes a self-cleaning tool 77 for facilitating the self-cleaning of the nasal irrigation device 1. The self-cleaning tool 77 includes a first conduit 78, a second conduit 79, and an intermediate chamber 80 that is fluidly coupled to both the first conduit 78 and the second conduit 79. In order to clean the nasal irrigation device 1, the self-cleaning tool 77 is coupled to the handle 15 and the tubing assembly 14. In particular, the tip of the first nasal insert 21 is inserted into the first conduit 78 and the tip of the second nasal insert 22 is inserted into the second conduit 79. The user then fills the clean liquid tank 12 with fresh water and a small amount (one or two drops) of liquid soap and switches the nasal irrigation device 1 on and allows it to run (by pressing the actuator 24) until the clean liquid tank 12 is empty. The wastewater from the cleaning process will fill the waste liquid tank 13 and may be disposed of by the user when the cleaning operation is completed.

While the presently disclosed technology has been described in detail and with reference to specific examples thereof, it will be apparent to one skilled in the art that various changes and modifications can be made therein without departing from the spirit and scope thereof. It is understood, therefore, that the presently disclosed technology is not limited to the particular embodiments disclosed, but it is intended to cover modifications within the spirit and scope of the presently disclosed technology.

What is claimed is:

1. A nasal irrigation device, comprising:
a clean liquid tank;
a waste liquid tank;
a tubing assembly;
a first nasal insert structured to be inserted into a first nostril of a user, the first nasal insert either being fixedly coupled to the tubing assembly or structured to be removably coupled to the tubing assembly;
a second nasal insert structured to be inserted into a second nostril of the user, the second nasal insert either being fixedly coupled to the tubing assembly or structured to be removably coupled to the tubing assembly;
a first motorized pump coupled to the clean liquid tank and the tubing assembly, the first motorized pump being structured to draw liquid from the clean liquid tank and pump the liquid to the tubing assembly and to the first nasal insert with positive pressure when the first nasal insert is fixedly or removably coupled to the tubing assembly; and
a second motorized pump separate from the first motorized pump and coupled to the waste liquid tank and the tubing assembly, the second motorized pump being structured to draw waste liquid from the second nasal insert and the tubing assembly with negative pressure when the second nasal insert is fixedly or removably coupled to the tubing assembly and provide the waste liquid to the waste liquid tank.

2. The nasal irrigation device according to claim 1, wherein the first motorized pump and the second motorized pump are structured and configured to simultaneously draw the liquid from the clean liquid tank and pump the liquid to the tubing assembly and the first nasal insert when the first nasal insert is coupled to the tubing assembly and draw the waste liquid from the tubing assembly and the second nasal insert when the second nasal insert is coupled to the tubing assembly and pump the waste liquid to the wastewater tank.

3. The nasal irrigation device according to claim 1, wherein the first motorized pump and the second motorized pump are each a centrifugal pump.

4. The nasal irrigation device according to claim 1, further comprising a main housing, wherein the clean liquid tank and the waste liquid tank are each structured to be selectively coupled to the main housing.

5. The nasal irrigation device according to claim 4, wherein the first motorized pump and the second motorized pump are provided within the main housing.

6. The nasal irrigation device according to claim 4, wherein the first motorized pump is fluidly coupled to the clean liquid tank through a first tube provided within the main housing and the second motorized pump is fluidly coupled to the waste liquid tank through a second tube provided within the main housing.

7. The nasal irrigation device according to claim 6, wherein the first tube is coupled to a female fluid coupling member provided at least partially within the main housing, the female fluid coupling member having a post member, wherein the clean liquid tank includes a male fluid coupling member extending from a front wall of the clean water tank that is structured to be received within the female fluid coupling member, wherein the male fluid coupling member includes a sealing ring provided on an outer surface of the male fluid coupling member structured to create a seal when the male fluid coupling member is received within the female fluid coupling member, wherein a spring and an activity pole are provided within the male fluid coupling member, wherein the spring biases the activity pole against a sealing surface provided at the terminal end of the male fluid coupling member, and wherein the post member is structured to engage the activity pole and push the activity pole to compress the spring and move the activity pole away from the sealing surface when the male fluid coupling member is received within the female fluid coupling member.

8. The nasal irrigation device according to claim 7, wherein the second tube is coupled to a second female fluid coupling member provided at least partially within the main housing, wherein the waste liquid tank includes a second male fluid coupling member extending from a front wall of the waste liquid tank that is structured to be received within the second female fluid coupling member, wherein the second male fluid coupling member includes a sealing ring provided on an outer surface of the second male fluid coupling member structured to create a seal when the second male fluid coupling member is received within the second female fluid coupling member.

9. The nasal irrigation device according to claim 8, wherein the male fluid coupling member, the female fluid coupling member, the second male fluid coupling member and the second female fluid coupling member each include a cylindrical cross-section.

10. The nasal irrigation device according to claim 8, wherein the male fluid coupling member is positioned immediately adjacent a bottom edge of the front wall of the clean liquid tank and the male fluid coupling member is positioned immediately adjacent a top edge of the front wall of the waste liquid tank.

11. The nasal irrigation device according to claim 10, wherein the rear wall of the main housing includes a pair of first slots for receiving a respective one of a pair of first tabs that are provided on the front wall of the clean liquid tank, wherein the male fluid coupling member is positioned in between the first slots, and wherein the rear wall of the main housing includes a pair of second slots for receiving a respective one of a pair of second tabs that are provided on a front wall of the waste liquid tank, wherein the second male fluid coupling member is positioned in between the second slots.

12. The nasal irrigation device according to claim 4, wherein a rear wall of the main housing includes a number of first slots for receiving a respective one of a number of first tabs that are provided on a front wall of the clean liquid tank, and wherein the rear wall of the main housing includes a number of second slots for receiving a respective one of a number of second tabs that are provided on a front wall of the waste liquid tank.

13. The nasal irrigation device according to claim 10, wherein the rear wall of the main housing includes a number of first support members extending outwardly from the rear wall for supporting a bottom surface of the clean liquid tank and a number of second support members extending outwardly from the rear wall for supporting a bottom surface of the waste liquid tank.

14. The nasal irrigation device according to claim 4, wherein the clean liquid tank and the waste liquid tank are each structured to be selectively coupled to the main housing in a manner wherein the clean liquid tank is positioned on top of the waste liquid tank.

15. The nasal irrigation device according to claim 14, wherein a top surface of the clean liquid tank includes an aperture for filling the clean liquid tank, wherein the clean liquid tank further includes a lid structured to be friction or snap fit onto the clean liquid tank to cover the aperture, wherein the lid includes a base and an upwardly extending and angled tab member.

16. The nasal irrigation device according to claim 4, wherein the clean liquid tank includes a rounded rear wall having a pair of elongated indentations for facilitating the coupling and de-coupling of the clean liquid tank to and from the main housing, and wherein the waste liquid tank includes a rounded rear wall having a pair of elongated indentations for facilitating the coupling and de-coupling of the waste liquid tank to and from the main housing.

17. The nasal irrigation device according to claim 1, wherein the tubing assembly includes a housing, a first tube, and a second tube, wherein a top end of the housing includes a first portion structured to receive and hold the first nasal insert and a second portion structured to receive and hold the second nasal insert, wherein the first tube is partially received within the housing and has a first end positioned outside of the housing and coupled to the first motorized pump and a second end contained within the housing and coupled to the first portion structured to receive and hold the first nasal insert, and wherein the second tube is partially received within the housing and has a first end positioned outside of the housing and coupled to the second motorized pump and a second end contained within the housing and coupled to the second portion structured to receive and hold the second nasal insert.

18. The nasal irrigation device according to claim 17, wherein the main housing include a compartment in a front side of the main housing and an opening providing access to the compartment, wherein the first tube, the second tube and a portion of the housing of the nasal tube assembly are structured to be removably received and held within the compartment in a manner wherein the top end of the housing of the nasal tube assembly is positioned outside of the main housing.

19. The nasal irrigation device according to claim 18, further comprising a first grip member extending upwardly from a first side of the top end of the housing of the nasal tube assembly and a second grip member extending upwardly from a second side of the top end of the housing of the nasal tube assembly, wherein the first portion structured to receive and hold the first nasal insert and the second portion structured to receive and hold the second nasal insert are positioned between the first grip member and the second grip member.

20. The nasal irrigation device according to claim 19, wherein the first grip member and the second grip member each include a plurality of teeth.

21. The nasal irrigation device according to claim 17, wherein the housing of the nasal tube assembly includes an actuator structured and configured to selectively activate and deactivate the first motorized pump and the second motorized pump when the nasal irrigation device is in a powered-on condition.

22. The nasal irrigation device according to claim 21, wherein the main housing includes a button structured and configured to move the nasal irrigation device into the powered-on condition and to selectively control and adjust a power level of the first motorized pump and the second motorized pump when the nasal irrigation device is in the powered-on condition.

23. The nasal irrigation device according to claim 22, wherein the power level of the first motorized pump and the second motorized pump is adjustable among three different levels using the button.

24. The nasal irrigation device according to claim 17, further comprising a self-cleaning tool for self-cleaning the nasal irrigation device using the nasal tube assembly and the first motorized pump and the second motorized pump, wherein the self-cleaning tool is structured to be selectively coupled to the nasal tube assembly.

25. The nasal irrigation device according to claim 24, wherein the self-cleaning tool includes a first conduit, a second conduit, and an intermediate chamber that is fluidly coupled to the first conduit and the second conduit, wherein the first conduit is structured to be coupled to the first nasal insert and the second conduit is structured to be coupled to the second nasal insert.

26. The nasal irrigation device according to claim 1, wherein the first nasal insert and the second nasal insert each include:
an elongated body member defining a channel extending through the body member along a longitudinal axis of the body member;
a first aperture defined at a distal end of the body member; and
an angled base provided at a proximal end of the body member and at an angle with respect to the longitudinal axis, wherein the angled base defines a second aperture in the body member.

27. The nasal irrigation device according to claim 26, wherein the angle is 45 degrees.

28. The nasal irrigation device according to claim 26, wherein the distal end is provided on a distal portion of the body member and the proximal end is provided on a proximal portion of the body member, wherein the proximal portion includes a skirt extending downwardly and outwardly from the distal portion of the body member, the skirt defining an annular space between a bottom edge of the skirt and a bottom of the proximal portion of the body.

29. The nasal irrigation device according to claim 28, wherein the bottom edge of the skirt includes a plurality of scallops.

30. The nasal irrigation device according to claim 28, wherein the distal portion has a cylindrical shape, wherein bottom of the proximal portion of the body has a cylindrical shape, and wherein a diameter of the distal portion is smaller than a diameter of the bottom of the proximal portion of the body.

31. The nasal irrigation device according to claim 4, wherein a rear wall of the main housing includes a number of shelves and a platform having a raised member extending from the rear wall, wherein a bottom of the clean liquid tank has a number of first indentations each sized to receive one of the number of shelves to facilitate coupling of the clean liquid tank to the main housing, and wherein a bottom of the waste liquid tank has a second indentation sized to receive the raised member to facilitate coupling of the waste liquid tank to the main housing.

32. The nasal irrigation device according to claim 4, wherein a bottom internal surface of the clean water tank lies in a first plane and a bottom of the main housing lies in a second plane, and wherein the first plane is tilted 2° relative to the second plane.

33. A method of providing nasal irrigation to an individual, comprising:
drawing clean liquid from a clean liquid tank and pumping the clean liquid to a first nostril of the individual with positive pressure generated by a first motorized pump while simultaneously drawing a waste liquid from a second nostril of the individual with negative pressure generated by a second motorized pump separate from the first motorized pump and providing the waste liquid to a waste liquid tank.
